(19) United States
(12) Patent Application Publication
(10) Pub. No.: US 2026/0191575 A1
(43) Pub. Date: Jul. 9, 2026

(54) DEVICES AND METHODS FOR SEPARATING A CATHETER FROM AN INTERNAL BONE FIXATION IMPLANT

(71) Applicant: IlluminOss Medical, Inc., East Providence, RI (US)

(72) Inventors: Gene P. DiPoto, Milford, MA (US); Robert A. Rabiner, Dortmund (DE); Anthony W. O'Leary, Walpole, MA (US)

(73) Assignee: IlluminOss Medical, Inc., East Providence, RI (US)

(21) Appl. No.: 19/442,672

(22) Filed: Jan. 7, 2026

Related U.S. Application Data
(60) Provisional application No. 63/742,651, filed on Jan. 7, 2025.

(51) Int. Cl. A61B 17/88; A61B 17/3205; A61M 25/00

(52) U.S. Cl. CPC A61B 17/8836; A61B 17/3205; A61M 25/0082

(57) ABSTRACT

Devices and methods for separating a catheter from an internal bone fixation implant are disclosed herein. According to aspects illustrated herein, there is provided a catheter cutter device comprises: a first tubular member having a proximal end, a distal end, and a lumen extending therethrough, wherein the proximal end of the first tubular member includes a threaded shank portion and a plain shank portion, wherein the plain shank portion of the first tubular member is configured to connect the first tubular member into a chuck of a drill, and wherein the distal end of the first tubular member terminates in a cutting head comprising a cutting blade; and a second tubular member having a proximal end, a distal end, and a lumen extending therethrough, wherein the proximal end of the second tubular member includes a handle having internal threads.

### RELATED APPLICATIONS

This application claims priority to and the benefit of U.S. Provisional Patent Application No. 63/742,651, filed Jan. 7, 2025, the contents of which is incorporated herein by reference in its entirety.

### FIELD

The embodiments disclosed herein relate to devices for separating a hardened internal bone fixation implant from an introducer catheter, and to methods of using these devices during a medical procedure.

### BACKGROUND

Bones form the skeleton of the body and allow the body to be supported against gravity and to move and function in the world. Bone fracture repair is the process of rejoining and realigning the ends of broken bones. Currently there are several internal approaches to repair, strengthen and support a fractured bone. One such device is the IlluminOss Photodynamic Bone Stabilization System (PBSS) developed by Illuminoss Medical, Inc. of East Providence, Rhode Island, USA, which is a treatment option for the fixation and stabilization of fractures through a minimally invasive procedure.

The PBSS includes an introducer catheter to deploy an expandable, noncompliant, balloon into a cleared-out medullary cavity across a fracture site. During the procedure, the balloon is infused with a photodynamic (light cured) monomer that causes the balloon to slowly expand and fill the intramedullary canal of the fractured bone. Activation of a light system allows for visible spectrum light to be delivered through a radially emitting light pipe that is temporarily positioned into a central lumen of the catheter that runs the length of the balloon. The liquid monomer within the balloon is exposed to light along the entire length of the balloon during the curing process. The hardened expandable balloon can then be separated from the introducer catheter using various techniques. The hardened expandable balloon remains within the void of the fractured bone and provides support and proper orientation of the fractured bone resulting in the repair, healing, and strengthening of the fractured bone.

A need remains for improved devices and methods for separating an internal bone fixation implant from an introducer catheter.

### SUMMARY

In some embodiments, a catheter cutter device comprises: a first tubular member having a proximal end, a distal end, and a lumen extending therethrough, wherein the proximal end of the first tubular member includes a threaded shank portion and a plain shank portion, wherein the plain shank portion of the first tubular member is configured to connect the first tubular member into a chuck of a drill, and wherein the distal end of the first tubular member terminates in a cutting head comprising a cutting blade; and a second tubular member having a proximal end, a distal end, and a lumen extending therethrough, wherein the proximal end of the second tubular member includes a handle having internal threads.

In some embodiments, the first tubular member is coaxially disposed within the lumen of the second tubular member so that the cutting head at the distal end of the first tubular member extends beyond the distal end of the second tubular member

In some embodiments, when the cutting head at the distal end of the first tubular member extends beyond the distal end of the second tubular member, the threaded shank portion of the first tubular member can mate with the internal threads of the handle of the second tubular member to form a secure mechanical connection.

In some embodiments, when the secure mechanical connection between the first tubular member and the second tubular member is formed, and the plain shank portion of the first tubular member is engaged with a drill, the first tubular member is configured to rotate and the second tubular member is configured to move along the first tubular members axis in a linear direction while not rotating.

In some embodiments, a method comprises: providing a catheter cutter device comprising: a first tubular member having a proximal end, a distal end, and a lumen extending therethrough, wherein the proximal end of the first tubular member includes a threaded shank portion and a plain shank portion, and wherein the distal end of the first tubular member terminates in a cutting head comprising a cutting blade; and a second tubular member having a proximal end, a distal end, and a lumen extending therethrough, wherein the proximal end of the second tubular member includes a handle having internal threads, wherein the first tubular member is coaxially disposed within the lumen of the second tubular member so that the cutting head at the distal end of the first tubular member extends beyond the distal end of the second tubular member, wherein the threaded shank portion of the first tubular member mates with the internal threads of the handle of the second tubular member to form a secure mechanical connection; positioning the cutting head of the catheter cutter device at a junction between an introducer catheter and a hardened expandable balloon, wherein the introducer catheter is coaxially disposed within the lumen of the first tubular member; connecting a drill to the plain shank portion of the first tubular member; powering on the drill causing the first tubular member to rotate and the second tubular member to move along the first tubular members axis in a linear direction while not rotating, the linear movement of the second tubular member pressing down on the cutting head of the first tubular member so the cutting blade punctures the introducer catheter as the cutting blade rotates around the introducer catheter; and separating the introducer catheter from the hardened expandable balloon.

In some embodiments, when starting the drill, the handle of the second tubular member is held by a user to prevent rotation of the second tubular member when the first tubular member is rotated.

In some embodiments, the method further comprises pulling back the catheter cutter device away from the separated hardened expandable balloon. In some embodiments, when pulling back the catheter cutter device away from the separated hardened expandable balloon, the lumen of the first tubular member includes the cut introducer catheter.

In some embodiments, a cutting head of a first tubular member of a catheter cutting device of the present disclosure comprises a lower support arm and an upper arm having the cutting blade, wherein the upper arm is configured to move from an open position to a closed positioned relative to the lower support arm.

In some embodiments, a kit comprises: an expandable balloon catheter; a biocompatible light curable liquid monomer; and a catheter cutter device, the catheter cutter device comprising: a first tubular member having a proximal end, a distal end, and a lumen extending therethrough, wherein the proximal end of the first tubular member includes a threaded shank portion and a plain shank portion, wherein the plain shank portion of the first tubular member is configured to connect the first tubular member into a chuck of a drill, and wherein the distal end of the first tubular member terminates in a cutting head comprising a cutting blade; and a second tubular member having a proximal end, a distal end, and a lumen extending therethrough, wherein the proximal end of the second tubular member includes a handle having internal threads, and wherein the threaded shank portion of the first tubular member is configured to mate with the internal threads of the handle of the second tubular member to form a secure mechanical connection. In some embodiments, the kit further comprises instructions for use.

In some embodiments, the first tubular member is coaxially disposed within the lumen of the second tubular member so that the cutting head at the distal end of the first tubular member extends beyond the distal end of the second tubular member

In some embodiments, when the cutting head at the distal end of the first tubular member extends beyond the distal end of the second tubular member, the threaded shank portion of the first tubular member can mate with the internal threads of the handle of the second tubular member to form a secure mechanical connection.

In some embodiments, when the secure mechanical connection between the first tubular member and the second tubular member is formed, and the plain shank portion of the first tubular member is engaged with a drill, the first tubular member is configured to rotate and the second tubular member is configured to move along the first tubular members axis in a linear direction while not rotating.

In some embodiments, a first tubular member and a second tubular member of a catheter cutting device of the present disclosure are manufactured from a medical-grade hypotube.

In some embodiments, a proximal end of a first tubular member of a catheter cutting device of the present disclosure is formed of an over-molded plastic part.

In some embodiments, a handle of a second tubular member of a catheter cutting device of the present disclosure is formed of an over-molded plastic part.

In some embodiments, a cutting head of a first tubular member of a catheter cutting device of the present disclosure is formed by laser cutting a distal end of a medical-grade hypotube.

In some embodiments, a cutting head of a first tubular member of a catheter cutting device of the present disclosure comprises a lower support arm and an upper arm having the cutting blade, wherein the upper arm is configured to move from an open position to a closed positioned relative to the lower support arm.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 illustrates an exemplary embodiment of a catheter cutter device comprising a first tubular member and a second tubular member;

FIG. 2 illustrates the catheter cutter device of FIG. 1 fully assembled, wherein the first tubular member is coaxially disposed within the second tubular member;

FIG. 3 illustrates a cross-sectional side view of the fully assembled catheter cutter device of FIG. 2;

FIG. 4 illustrates a close-up side view of a distal end of the fully assembled catheter cutter device of FIG. 2;

FIG. 5 illustrates a close-up perspective view of the catheter cutter device of FIG. 4;

FIG. 6 illustrates a close-up top view of the catheter cutter device of FIG. 4;

FIG. 7 illustrates a close-up perspective view of a portion of the catheter cutter device of FIG. 4;

FIG. 8 illustrates the fully assembled catheter cutter device of FIG. 2 engaged with a power drill;

FIGS. 9A-9D illustrate embodiments of second tubular members that are configured to be used with a first tubular member as part of a catheter cutter device of the present disclosure;

FIGS. 10A-10D illustrate embodiments of first tubular members that are configured to be used with a second tubular member as part of a catheter cutter device of the present disclosure;

FIGS. 11A-11D illustrate embodiments of first tubular members that are configured to be used with a second tubular member as part of a catheter cutter device of the present disclosure;

FIG. 12 illustrates a close-up side view of an exemplary embodiment of an introducer catheter having an expandable balloon at a distal end thereof;

FIG. 13 illustrates an exemplary embodiment of the catheter cutter device of FIG. 2 coaxially surrounding the introducer catheter from FIG. 12;

FIG. 14 illustrates an exemplary embodiment of the catheter cutter device from FIG. 13 in position prior to separating the hardened expanded balloon from the introducer catheter, wherein a power drill engages the plain shank portion at the proximal end of the first tubular member;

FIG. 15 illustrates an exemplary embodiment of the catheter cutter device from FIG. 14 after separating the hardened expanded balloon from the introducer catheter;

FIG. 16 illustrates an exemplary embodiment of the catheter cutter device of FIG. 2 in use during a medical procedure for repairing a fractured humerus bone; and

FIG. 17 illustrates an exemplary embodiment of a catheter cutter device comprising a flexible first tubular member and a flexible second tubular member.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Currently there are several internal approaches to repair, strengthen and support a fractured bone. One such device is the IlluminOss Photodynamic Bone Stabilization System (PBSS) developed by Illuminoss Medical, Inc. of East Providence, Rhode Island, USA, which is a treatment option for the fixation and stabilization of fractures through a minimally invasive procedure. In some embodiments, a PBSS is comprised of a single use disposable procedure pack, along with a reusable curing system and instrument set. The PBSS includes an introducer catheter with a small diameter expandable, noncompliant, balloon, wherein the small diameter expandable, noncompliant, balloon is positioned into a cleared-out medullary cavity across a fracture site. During the procedure, once in correct alignment and position, the balloon is infused with a photodynamic (light cured) liquid monomer that causes the balloon to slowly expand and fill the intramedullary canal of the fractured bone. Activation of a light system allows for visible spectrum light to be delivered through a radially emitting light pipe that is temporarily positioned into a central lumen of the introducer catheter that runs the length of the balloon. The liquid monomer within the balloon is exposed to light along the entire length of the balloon during the curing process. The hardened expandable balloon can then be separated from the introducer catheter using various techniques. The hardened expandable balloon remains within the void of the fractured bone and provides support and proper orientation of the fractured bone resulting in the repair, healing, and strengthening of the fractured bone. A hardened internal bone fixation implant of the kind mentioned is known for example from U.S. Pat. Nos. 7,806,900 and 8,574,233, 8,939,977, and U.S. Pat. No. 9,433,450.

At the end of the medical procedure for internal bone fixation using a PBSS as described above, the introducer catheter is required to be separated from the hardened internal bone fixation implant. Aspects of the present disclosure relate to such a separation device, herein referred to as a catheter cutter device. By way of a non-limiting example, the devices and methods of the present disclosure are described in connection with separating a hardened expandable balloon from an introducer catheter. The devices and methods of the present disclosure may be applicable for separating other types of internal bone fixation implants from suitable introducers.

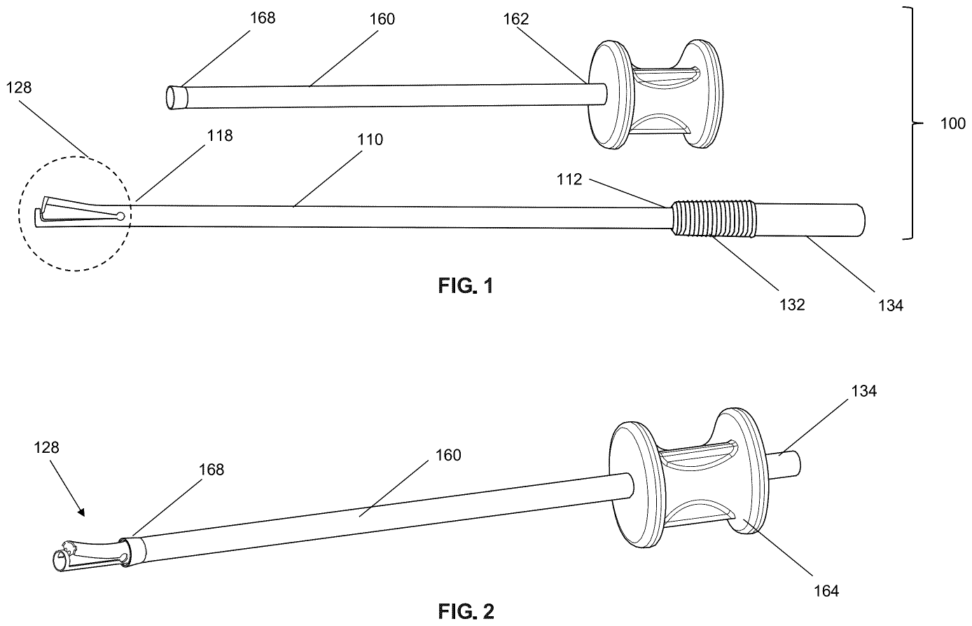

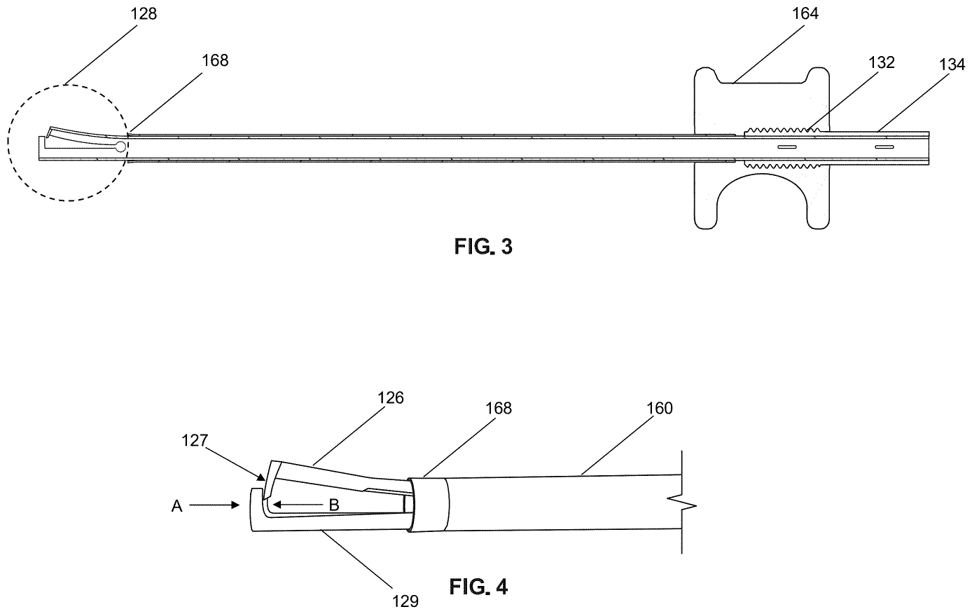

FIG. 1, in conjunction with FIG. 2, illustrates an exemplary embodiment of a catheter cutter device 100 comprising a first tubular member 110 and a second tubular member 160. First tubular member 110 includes a proximal end 112, a distal end 118, and a lumen 120 extending therethrough. Proximal end 112 includes a threaded shank portion 132 and a plain shank portion 134. Distal end 118 includes a cutting head 128 as described in more detail below. Second tubular member 160 includes a proximal end 162, a distal end 168, and a lumen 170 therebetween. Proximal end 162 includes a handle 164 having internal threads 166, as best illustrated in the cross-sectional view shown in FIG. 3. In some embodiments, the first tubular member 110 and the second tubular member 160 are manufactured from a medical-grade hypotube. In some embodiments, the medical-grade hypotube is a thin-walled metal tube manufactured from a material including 304 and 316 stainless steel, nitinol, 17-4, 17-7, and cobalt chrome (MP35N and L605), and combinations thereof. In some embodiments, handle 164 of the second tubular member 160 and proximal end 112 of the first tubular member 110 can be formed of an over-molded plastic part. In some embodiments, the first tubular member 110 is longer than the second tubular member 160.

The size/diameter of a catheter cutter device of the present disclosure can vary. The lumen 120 of the first tubular member 110 should have a diameter that is large enough to be positioned coaxially over an introducer catheter of a PBSS. In some embodiments, the diameter of the lumen 120 of the first tubular member 110 can be between 2 mm and 10 mm. The size/diameter of an introducer catheter of a PBSS can vary. Each introducer catheter of different diameters can have different catheter cutter devices as the catheter cutter device is introducer catheter diameter specific.

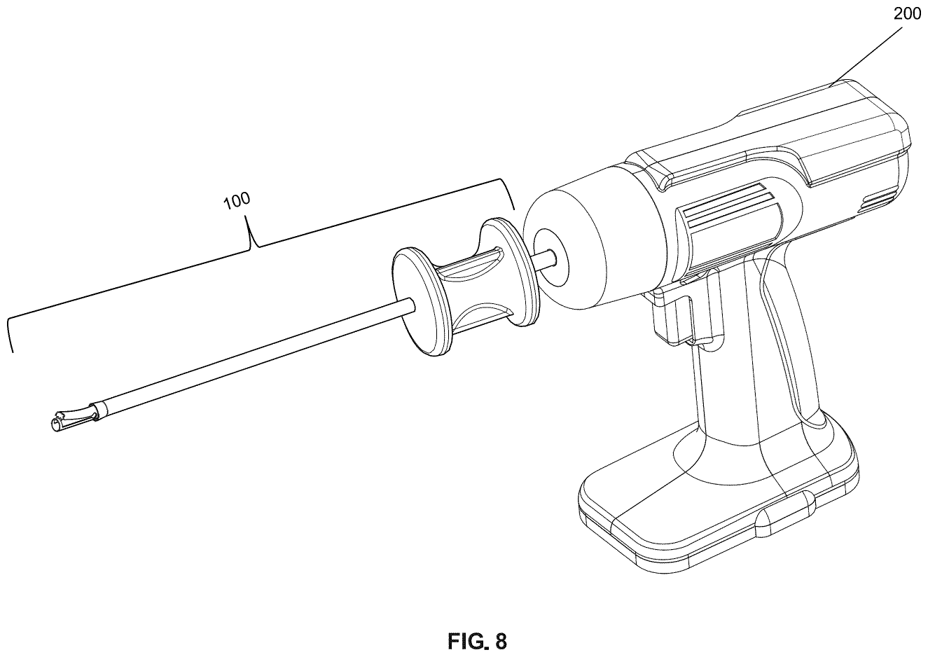

As shown in FIG. 2, in conjunction with FIG. 3, the first tubular member 110 is coaxially disposed within the second lumen 170 of the second tubular member 160 such that the cutting head 128 at the distal end 118 of the first tubular member 110 extends beyond the distal end 168 of the second tubular member 160. When the first tubular member 110 is coaxially disposed within the second lumen 170 of the second tubular member 160, the threaded shank portion 132 of the first tubular member 110 can mate with the internal threads 166 of the handle 164 of the second tubular member 160 to form a secure mechanical connection. When the secure mechanical connection between the first tubular member 110 and the second tubular member 160 is formed, and the proximal plain shank portion 134 of the first tubular member 110 is engaged with a drill 200, as shown in FIG. 8 for example, the first tubular member 110 can rotate, the second tubular member 160 cannot rotate, and therefore the second tubular member 160 moves along the first tubular members 110 axis in a linear direction. The proximal plain shank portion 134 of the first tubular member 110 can include one, two, three, or more sections making up the plain shank portion 134, and may be designed for ease of attachment with a drill chuck. In some embodiments, the proximal plain shank portion 134 has three equally spaced flats that easily engage with the three jaws of a chuck. Other similar quick disconnect features may also be used on the proximal plain shank portion 134 to align with quick disconnect mechanisms of a drill are within the scope of the present disclosure.

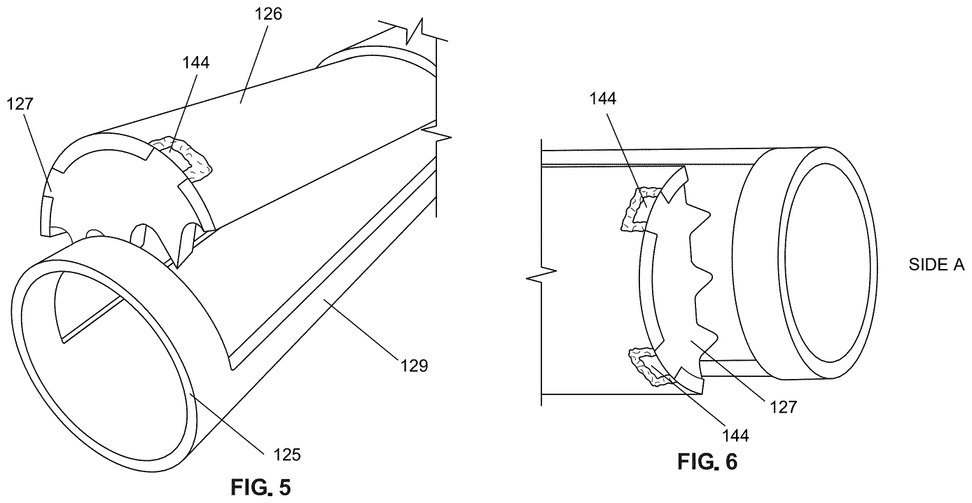

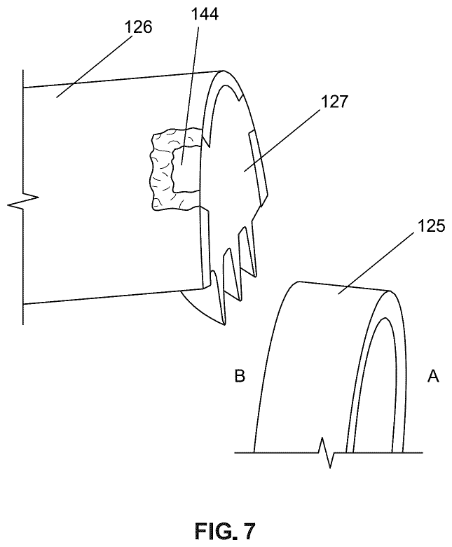

FIG. 4, FIG. 5, FIG. 6, and FIG. 7 illustrate close-up views of the distal ends 118, 168 of the first tubular member 110 and the second tubular member 170. In some embodiments, the first tubular member 110 is formed from an equal diameter medical-grade hypotube that has been customized to include the cutting head 128. In some embodiments, the cutting head 128 at the distal end 118 of the first tubular member 110 is formed using laser cutting. For example, in some embodiments the cutting head 128 is formed by laser cutting into an upper surface of the equal diameter medical-grade hypotube at a given distance from a distal tip of the distal end 118 and then cutting into the central axis of the first lumen 120 a distance towards the proximal end 112. This leaves the distal tip of the distal end 118 uncut and creates a ring assembly 125 and a lower support arm 129. The cut-into upper surface results in an upper arm 126. The lower support arm 129 is positioned substantially opposite the upper arm 126 and a point of flexibility 121 is created, as best seen in FIG. 1. A cutting blade 127 can be joined to the upper arm 126. In some embodiments, the cutting blade 127 is laser welded to the upper arm 126 at one or more areas 144, as best seen in FIGS. 5 and 6. Cutting blade 127 engages the upper arm 126 in a perpendicular manner so that when the upper arm 126 is pushed down or compressed towards the lower support arm 129, the cutting blade 127 is on side “B” of the ring assembly 125, as best seen in FIG. 7, for example. During use, side “A” of ring assembly 125 is positioned at a junction between an introducer catheter and a hardened expandable balloon, as will be described in more detail below. In some embodiments, the ring assembly 125 creates a barrier between the hardened expandable balloon and the cutting blade 127. The ring assembly 125 can be used to prevent the first tubular member 110 from moving distally to the location of the hardened expandable balloon and cutting the hardened expandable balloon rather than the introducer catheter. Thus, the ring assembly 125 can act as a forward-motion stabilizing device to prevent distal motion of the cutting blade 127 such that the cutting blade 127 cannot be advanced further than desired.

In alternate embodiments, the first tubular member can include an upper arm and a lower support arm, wherein both arms include blades. The opposed first and second blades can cut through an introducer catheter in two different locations.

To fully assemble the catheter cutter device 100, the first tubular member 110 is positioned within the second lumen 170 of the second tubular member 160 so that the cutting head 128 at the distal end 118 of the first tubular member 110 extends beyond the distal end 168 of the second tubular member 160, and the threaded shank portion 132 of the first tubular member 110 mates with the internal threads 166 of the handle 164 of the second tubular member 160 to form a secure mechanical connection. The plain shank portion 134 of the first tubular member 110 is ready to be engaged with a drill. Once the drill is powered on, the drill spins the first tubular member 110, with the threads driving the second tubular member 160 forward when held by hand at the handle 164. When the second tubular member 160 reaches its maximum forward position, the internal threaded portion 166 is stopped. In some embodiments, threads terminate in a circle, only permitting the cutting head 128 to rotate in the same position, preventing unrestricted motion.

In some embodiments, the distal end 168 of the second tubular member 160 is flared, as seen, for example, in FIG. 4. In some embodiments, the flare is designed to allow a “feed-in” transition that compresses the cutting blade 127 of the first tubular member 110 smoothly as the second tubular member 170 moves distally.

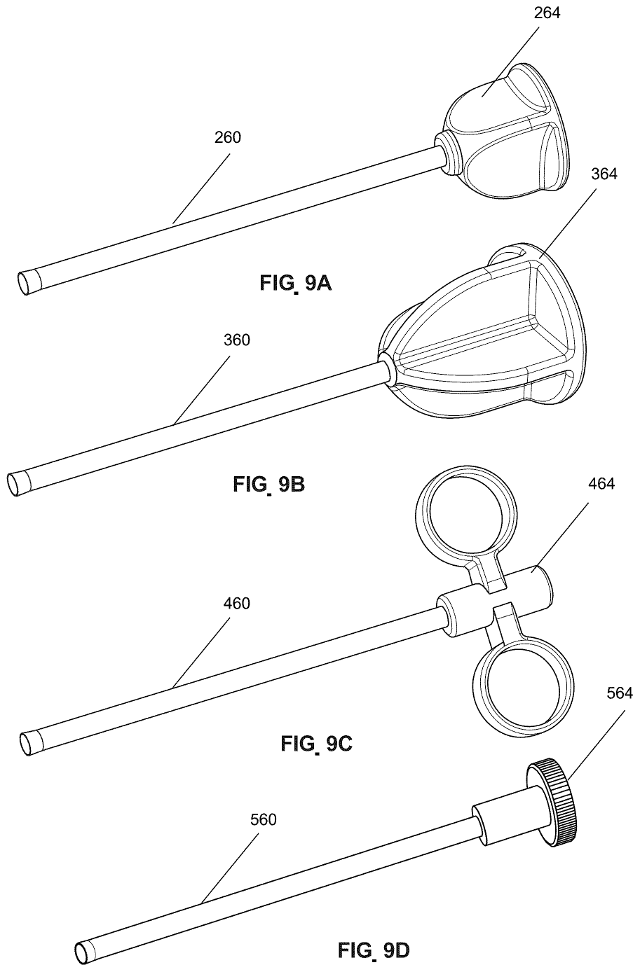

FIGS. 9A-9D illustrate embodiments of second tubular members 260, 360, 460 and 560 that are configured to be used with a first tubular member of the present disclosure as part of a catheter cutter device of the present disclosure. Each of second tubular members 260, 360, 460 and 560 have different style handles 264, 364, 464, and 564, respectively. A user can select a second tubular member based on their handle preference. All other features of the second tubular members 260, 360, 460 and 560 are consistent with those described above with respect to second tubular member 160.

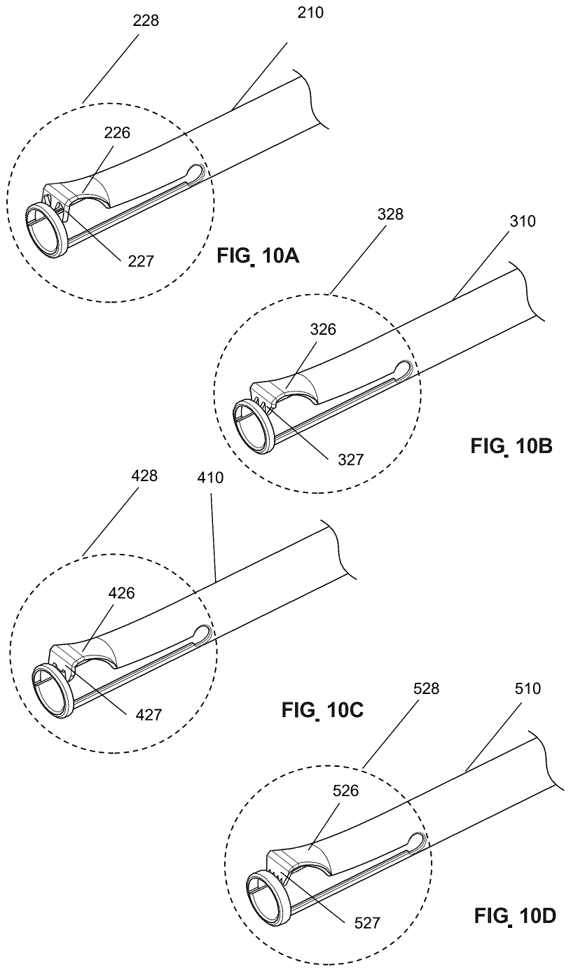

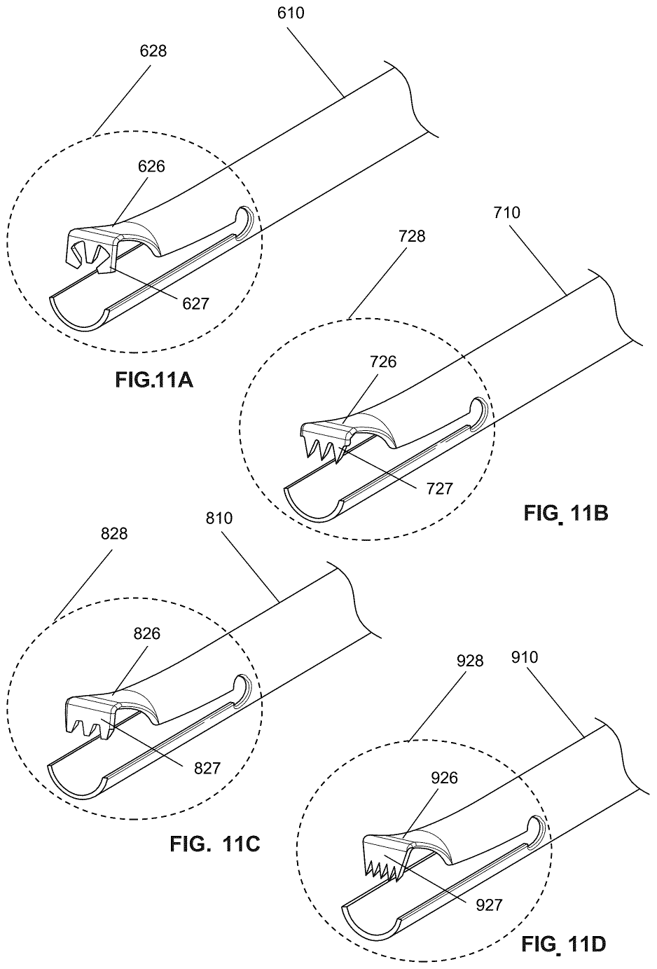

FIGS. 10A-10D illustrate embodiments of first tubular members 210, 310, 410, and 510, that are configured to be used with a second tubular member of the present disclosure as part of a catheter cutter device of the present disclosure. Each of first tubular members 210, 310, 410, and 510 have different cutting heads 228, 328, 428, and 528 that include upper arms 226, 326, 426, and 526 with cutting blades 227, 327, 427, and 527, respectively. The size, shape, edge configurations of the cutting blades 227, 327, 427, and 527 can vary. All other features of the first tubular members 210, 310, 410, and 510 are consistent with those described above with respect to first tubular member 110.

FIGS. 11A-11D illustrate embodiments of first tubular members 610, 710, 810, and 910, that are configured to be used with a second tubular member of the present disclosure as part of a catheter cutter device of the present disclosure. Each of first tubular members 610, 710, 810, and 910 have different cutting heads 628, 728, 828, and 928 that include upper arms 626, 726, 826, and 926 with cutting blades 627, 727, 827, and 927, respectively. The size, shape, edge configurations of the cutting blades 627, 727, 827, and 927 can vary. In the embodiments depicted in FIGS. 11A-11D, there is no ring assembly at lower support arms 629, 729, 829, and 929. All other features of the first tubular members 610, 710, 810, and 910 are consistent with those described above with respect to first tubular member 110.

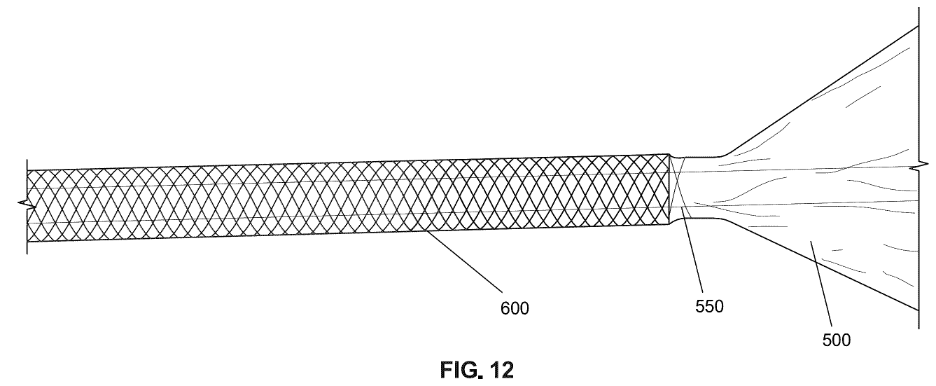

FIG. 12 illustrates a close-up side view of an exemplary embodiment of an introducer catheter 600 having an expandable balloon 500 at a distal end thereof. A neck 550 of the expandable ballon 500 is connected to the inner diameter of the introducer catheter 600. The fact that the expandable ballon 500 is within the introducer catheter 600 creates a natural step/fall off in diameter from the outer introducer catheter 600 dimension to the outer diameter dimension of the expandable ballon neck 550. This step creates a natural alignment/registration point for a catheter cutter device of the present disclosure to maintain position at the desired point of transection.

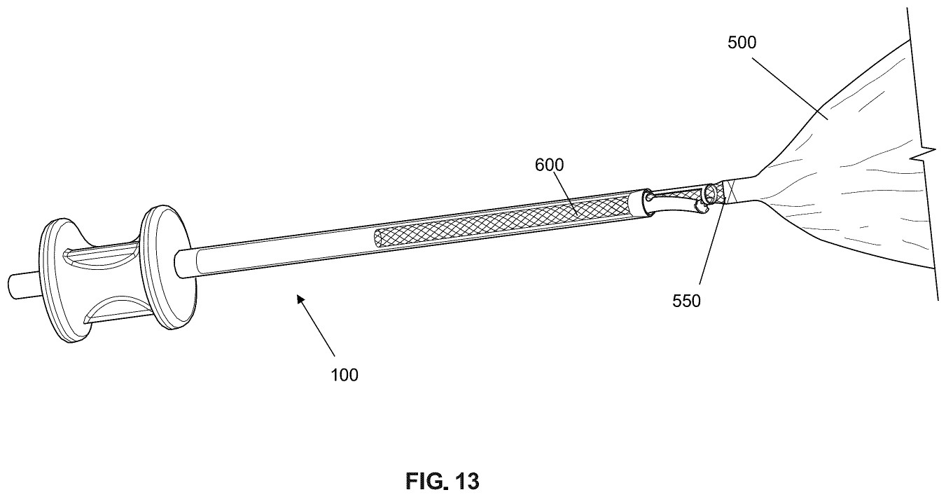

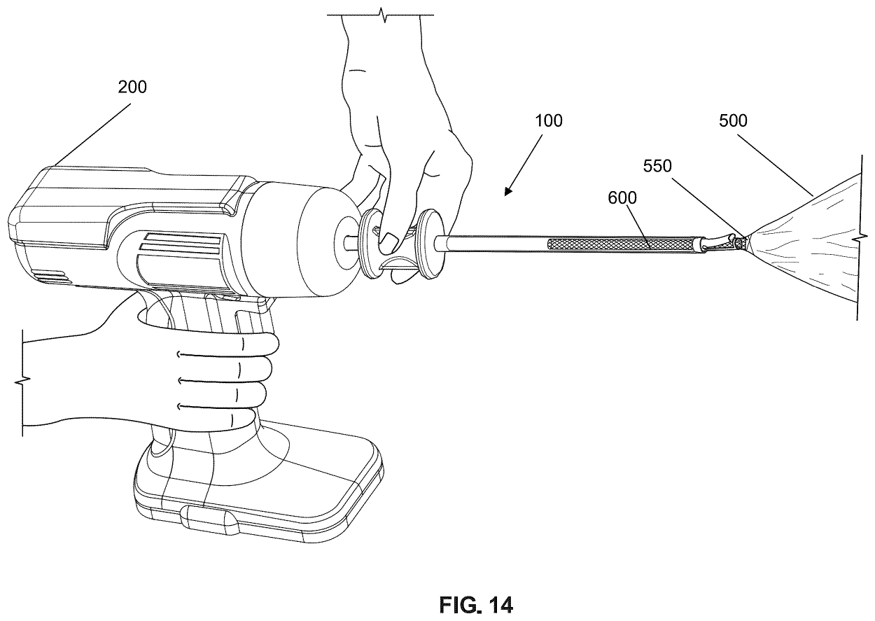

FIG. 13 illustrates an exemplary embodiment of the introducer catheter 600 coaxially disposed within the lumen 120 of the first tubular member 110, and the catheter cutter device 100 being longitudinally displaceable with respect to the introducer catheter 600. The cutting head 128 of the catheter cutter device 100 is positioned near neck 550. FIG. 14 illustrates a drill 200 being connected to the plain shank portion 134 of the first tubular member 110 and a user holding the handle 164 of the second tubular member 160 in position.

Forward rotation of the drill 200 automatically translates the second tubular member 160 distally over the first tubular member 110 when the second tubular member 160 is held in position by hand or other means. This translation of the second tubular member 160 creates downward pressure on the rotating cutting blade 127 of cutting head 128 to initiate cutting through the introducer catheter 600 at the neck 550. In some embodiments, with forward rotation of the drill 200, the thread design is left-handed to create linear translation of the second tubular member 160 from proximal to distal when the drill 200 is rotated in the forward rotational direction (clockwise viewed from behind the drill). Conversely, if a standard right-handed thread mechanism for the threaded engagement between the first tubular member 110 and the second tubular member 160 is used, then the proximal to distal translation of the second tubular member 160 could only occur with reverse rotation of the drill 200 (counter-clockwise viewed from behind the drill).

Various methodologies can be used to determine when the introducer catheter 600 is completely separated from the hardened expanded balloon 500. In some embodiments, there is a tactile feel of the separation of the introducer catheter 600. For example, an introducer catheter 600, at the time of the separation from the hardened expanded balloon 500, has a small wall thickness, a thin layer of cured monomer, and a thin wall of a guide tube (the tube that houses the light fiber) that separates the inner lumen of the introducer catheter 600. When the catheter cutter device 100 transects the walls to cut into the inner lumen of the insertion catheter 600, the user can feel the separation.

The number of rotations of the first tubular member to facilitate cutting of the introducer catheter can vary. In some embodiments, approximately 15-30 revolutions of the cutting head 127 on the first tubular member, with constant pressure, can be used towards a cut/separation. In some embodiments, approximately 16-20 revolutions of the cutting head 127 on the first tubular member, with constant pressure, can be used towards a cut/separation. In some embodiments, approximately 20-30 revolutions of the cutting head 127 on the first tubular member, with constant pressure, can be used towards a cut/separation. In some embodiments, it will depend on the pitch of the threads. For example, with constant pressure, it can be used towards a cut/separation. In some embodiments, ⅜″-16 internal threads 166 is used to provide a reasonable balance between power drill rotational speed and linear translation of the second tubular member 160. A rotational speed of the power drill between approximately 100-200 RPM translates the second tubular member 160 distally about an inch and the introducer catheter can be cut off and removed in less than 15 seconds. In some embodiments, the ⅜″-16 internal threads 166 is also sized larger than the first tubular member 110 to permit a preferred over-molding of threaded shank portion 132 onto the proximal end 112 of first tubular member 110. Other thread sizes larger than the OD of the first tubular member 110 may be utilized. Various thread pitches (TPI, threads per inch) may also be utilized to speed up or slow down the linear translation of the second tubular member 160. The internal threads 166 may be constructed from a variety of materials including plastics (polyacetal, PEEK, polystyrene, polycarbonate, ABS etc.) and metals (aluminum, stainless steel, etc.).

In some embodiments, a drill is not used as the drive mechanism. In some embodiments, the drive mechanism is in the form of a handle system that forces a second tubular member forward over a spinning first tubular member.

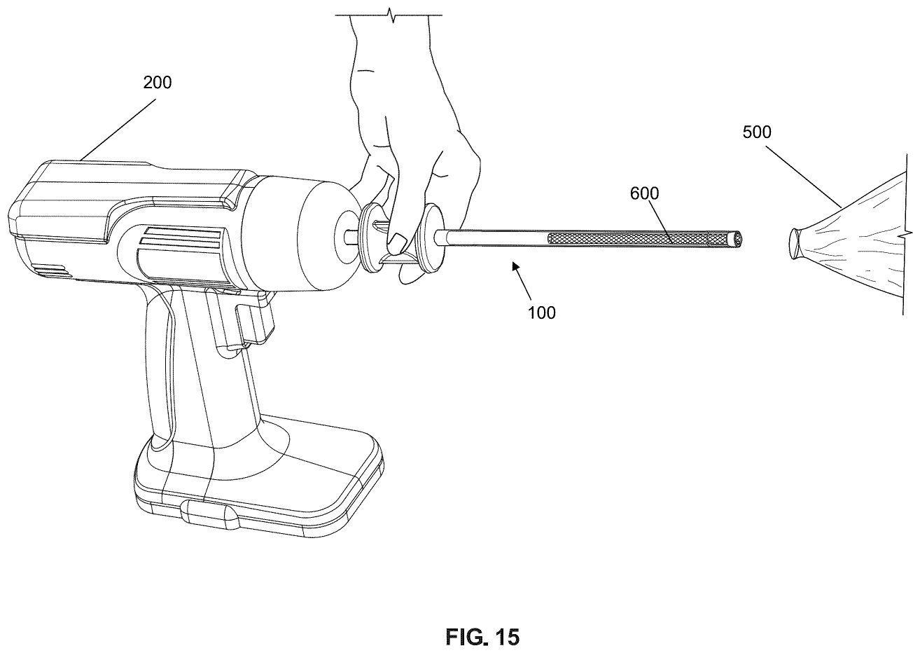

FIG. 15 illustrates an exemplary embodiment wherein the introducer catheter 600 has been completely separated from the hardened expanded balloon 500. The catheter cutter device 100 captures the introducer catheter 600 so there isn't risk of the introducer catheter 600 getting lost in the incision and leaves the hardened expanded balloon 500 alone.

A catheter cutter device of the present disclosure can be used in a medical procedure for treating traumatic, fragility, pathological, and impending pathological fractures of the humerus, radius, ulna, clavicle, pelvis, fibula, metacarpals, metatarsals, and phalanges with a photodynamic bone stabilization system (PBSS), such as the IlluminOss Photodynamic Bone Stabilization System (PBSS) developed by Illuminoss Medical, Inc. of East Providence, Rhode Island, USA. In some embodiments, a kit for use during the medical procedure includes an expandable balloon catheter; a biocompatible light curable liquid monomer; and a catheter cutter device of the present disclosure. In some embodiments, the kit further comprises instructions for use. In some embodiments, the expandable balloon catheter is a small-diameter expandable balloon at a distal end of an introducer catheter. In some embodiments, a proximal end of the introducer catheter includes a proximal end adapter having at least one arm and at least one adapter which can be utilized for the infusion and withdrawal of fluids or as conduits for the introduction of devices (e.g., a light-conducting fiber). In some embodiments, an adapter is a Luer lock. In some embodiments, an adapter is a Tuohy-Borst connector. In some embodiments, an adapter is a multi-functional adapter. In some embodiments, the proximal end of the introducer catheter is a three-arm proximal end fitting having a first adapter, a second adapter, and a third adapter. The first adapter can accept, for example, a light-conducting fiber configured to communicate light from a light source within an inner lumen that passes through the longitudinal axis of the flexible introducer catheter and the expandable balloon. The second adapter can accept, for example, air or fluid. The third adapter can accept, for example, a syringe housing the biocompatible light curable liquid monomer.

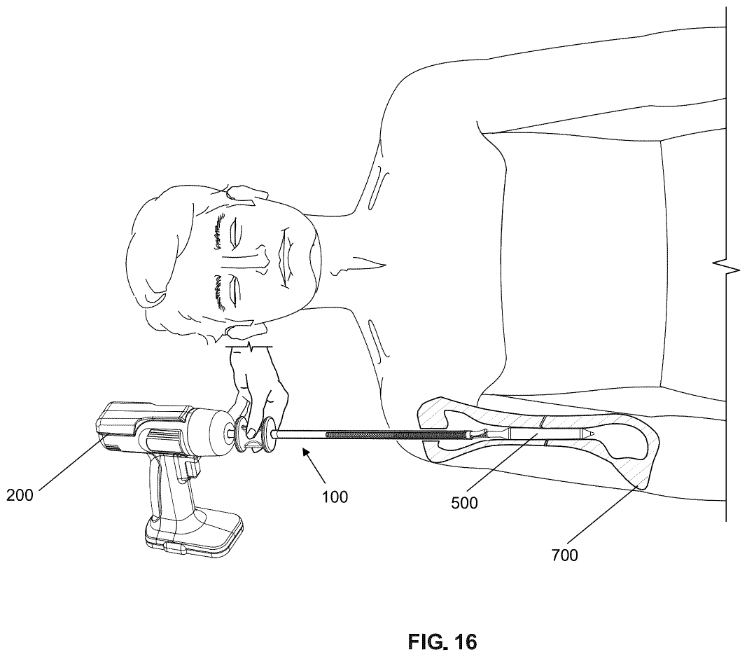

FIG. 16 illustrates an exemplary embodiment of the catheter cutter device 100 in use during a medical procedure for repairing a fractured humerus bone 700. In some embodiments, the following steps are performed for implanting an expandable balloon 500 of an intramedullary implant within the intramedullary space of a weakened or fractured humerus bone 700. A minimally invasive incision may be made through the skin of the patient's body to expose a fractured bone 700. The incision may be made at the proximal end or the distal end of the fractured bone 700 to expose the bone surface. Once the bone 700 is exposed, it may be necessary to retract some muscles and tissues that may be in view of the bone 700. An access hole can be formed in the bone700 by drilling or other methods known in the art. In some embodiments, the access hole has a diameter of about 3 min to about 10 mm. In some embodiments, the access hole has a diameter of about 3 mm.

The access hole extends through a hard compact outer layer of the bone 700 into the relatively porous inner of cancellous tissue. For bones with marrow, the medullary material should be cleared from the medullary cavity prior to insertion of the expandable balloon catheter. Once the medullary cavity is reached, the medullary material including air, blood, fluids, fat, marrow, tissue and bone debris should be removed to form a void. The void is defined as a hollowed out space, wherein a first position defines the most distal edge of the void with relation to the penetration point on the bone, and a second position defines the most proximal edge of the void with relation to the penetration site on the bone. The bone 700 may be hollowed out sufficiently to have the medullary material of the medullary cavity up to the cortical bone removed. There are many methods for removing the medullary material that are known in the art and within the spirit and scope on the presently disclosed embodiments.

A guidewire may be introduced into the bone via the access hole and placed between bone fragments of the bone to cross the location of a fracture. The guidewire may be delivered into the lumen of the bone and may cross the location of the break so that the guidewire spans multiple sections of bone fragments. The expandable balloon 500 (for example, a PET balloon) of the introducer catheter 600 is delivered to the site of the fracture and spans the bone fragments of the bone 700. In some embodiments, a guidewire may be placed into the intramedullary cavity of the bone and a sheath, with assistance from a dilator, combination may be advanced over the guidewire. Once the sheath is inside the intramedullary cavity, the guidewire and dilator can be withdrawn. Next, the expandable balloon 500 may be placed into a sheath, which can be removed to leave the expandable balloon 500 in place inside the intramedullary cavity. In some embodiments, the location of the expandable balloon 500 may be determined using at least one radiopaque marker which is detectable from the outside or the inside of the bone. Once the expandable balloon 500 is in the correct position within the fractured bone 700, a delivery system, such as a syringe, which contains biocompatible light-sensitive light curable monomer is attached to an adapter at a proximal end of the introducer catheter 600. The light-sensitive light curable monomer is then infused through the inner lumen in the introducer catheter 600 and enters the inner cavity of the expandable balloon 500. This addition of the light-sensitive light curable monomer within the expandable balloon 500 causes the expandable balloon 500 to expand. As the expandable balloon 500 is expanded, the fracture is reduced. Unlike traditional implants, such as rods, that span the fracture site, the expandable balloon 500 does more than provide longitudinal strength to both sides of the fractured bone 700. In some embodiments, the expandable balloon 500 can be a spacer for reducing the fracture and for holding the fractured and compressed bones apart at the point of the collapsed fracture.

Once orientation of the bone fragments are confirmed to be in a desired position, the light-sensitive light curable monomer may be hardened within the expandable balloon 500, such as by illumination with a visible emitting light source (for example, a light source having a wavelength of 436 nm). After the light-sensitive light curable monomer has been hardened, the light source may be removed from the expandable balloon catheter. Alternatively, the light source may remain in the expandable balloon 500 to provide increased rigidity.

The expandable balloon 500, once hardened, may be released from the introducer catheter 600 using a catheter cutter device 100 of the present disclosure to form a customized intramedullary rod (internal bone fixation implant) inside the intramedullary cavity of the bone 700. The hardened expandable balloon 500, which conforms to the anatomic contours of the medullary canal making it a customized intramedullary rod, provides longitudinal and rotational stability to the affected bone.

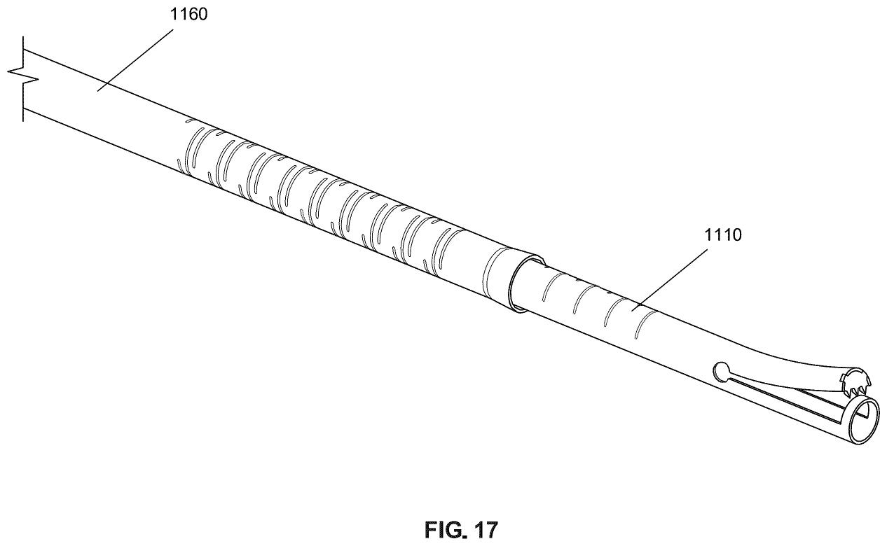

FIG. 17 illustrates an exemplary embodiment of a catheter cutter device 1000 comprising a first flexible tubular member 1110, and a second flexible tubular member 1160. In some embodiments, one or more cuts are made in the first flexible tubular member 1110 and the second flexible tubular member 1160. For example, one or more laser cuts can be made. The cuts are made perpendicular to the axis of the tubular members to allow deflection. Thus, as the cutter device 1000 is being positioned over an introducer catheter, the tubular members can follow the path of the introducer catheter and bend if necessary. This allows both the introducer catheter and the catheter cutter device 1000 to bend in the same fashion/radius. In some embodiments, this can allow the second flexible tubular member 1160 to be advanced over the first flexible tubular member 1110, even while they are both flexed. All other features of the flexible first tubular member 1110 and the flexible second tubular member 1160 can be similar to the embodiment described above.

In some embodiments, a catheter cutter device comprises: a first tubular member having a proximal end, a distal end, and a lumen extending therethrough, wherein the proximal end of the first tubular member includes a threaded shank portion and a plain shank portion, wherein the plain shank portion of the first tubular member is configured to connect the first tubular member into a chuck of a drill, and wherein the distal end of the first tubular member terminates in a cutting head comprising a cutting blade; and a second tubular member having a proximal end, a distal end, and a lumen extending therethrough, wherein the proximal end of the second tubular member includes a handle having internal threads.

In some embodiments, the first tubular member is coaxially disposed within the lumen of the second tubular member so that the cutting head at the distal end of the first tubular member extends beyond the distal end of the second tubular member

In some embodiments, when the cutting head at the distal end of the first tubular member extends beyond the distal end of the second tubular member, the threaded shank portion of the first tubular member can mate with the internal threads of the handle of the second tubular member to form a secure mechanical connection.

In some embodiments, when the secure mechanical connection between the first tubular member and the second tubular member is formed, and the plain shank portion of the first tubular member is engaged with a drill, the first tubular member is configured to rotate and the second tubular member is configured to move along the first tubular members axis in a linear direction while not rotating.

In some embodiments, a method comprises: providing a catheter cutter device comprising: a first tubular member having a proximal end, a distal end, and a lumen extending therethrough, wherein the proximal end of the first tubular member includes a threaded shank portion and a plain shank portion, and wherein the distal end of the first tubular member terminates in a cutting head comprising a cutting blade; and a second tubular member having a proximal end, a distal end, and a lumen extending therethrough, wherein the proximal end of the second tubular member includes a handle having internal threads, wherein the first tubular member is coaxially disposed within the lumen of the second tubular member so that the cutting head at the distal end of the first tubular member extends beyond the distal end of the second tubular member, wherein the threaded shank portion of the first tubular member mates with the internal threads of the handle of the second tubular member to form a secure mechanical connection; positioning the cutting head of the catheter cutter device at a junction between an introducer catheter and a hardened expandable balloon, wherein the introducer catheter is coaxially disposed within the lumen of the first tubular member; connecting a drill to the plain shank portion of the first tubular member; powering on the drill causing the first tubular member to rotate and the second tubular member to move along the first tubular members axis in a linear direction while not rotating, the linear movement of the second tubular member pressing down on the cutting head of the first tubular member so the cutting blade punctures the introducer catheter as the cutting blade rotates around the introducer catheter; and separating the introducer catheter from the hardened expandable balloon.

In some embodiments, when starting the drill, the handle of the second tubular member is held by a user.

In some embodiments, the method further comprises sliding the catheter cutter device away from the separated hardened expandable balloon. In some embodiments, when sliding the catheter cutter device away from the separated hardened expandable balloon, the lumen of the first tubular member includes the introducer catheter.

In some embodiments, a cutting head of a first tubular member of a catheter cutting device of the present disclosure comprises a lower support arm and an upper arm having the cutting blade, wherein the upper arm is configured to move from an open position to a closed positioned relative to the lower support arm.

In some embodiments, a kit comprises: an expandable balloon catheter; a biocompatible light curable liquid monomer; and a catheter cutter device, the catheter cutter device comprising: a first tubular member having a proximal end, a distal end, and a lumen extending therethrough, wherein the proximal end of the first tubular member includes a threaded shank portion and a plain shank portion, wherein the plain shank portion of the first tubular member is configured to connect the first tubular member into a chuck of a drill, and wherein the distal end of the first tubular member terminates in a cutting head comprising a cutting blade; and a second tubular member having a proximal end, a distal end, and a lumen extending therethrough, wherein the proximal end of the second tubular member includes a handle having internal threads, and wherein the threaded shank portion of the first tubular member is configured to mate with the internal threads of the handle of the second tubular member to form a secure mechanical connection. In some embodiments, the kit further comprises instructions for use.

In some embodiments, the first tubular member is coaxially disposed within the lumen of the second tubular member so that the cutting head at the distal end of the first tubular member extends beyond the distal end of the second tubular member

In some embodiments, when the cutting head at the distal end of the first tubular member extends beyond the distal end of the second tubular member, the threaded shank portion of the first tubular member can mate with the internal threads of the handle of the second tubular member to form a secure mechanical connection.

In some embodiments, when the secure mechanical connection between the first tubular member and the second tubular member is formed, and the plain shank portion of the first tubular member is engaged with a drill, the first tubular member is configured to rotate and the second tubular member is configured to move along the first tubular members axis in a linear direction while not rotating.

In some embodiments, a first tubular member and a second tubular member of a catheter cutting device of the present disclosure are manufactured from a medical-grade hypotube.

In some embodiments, a proximal end of a first tubular member of a catheter cutting device of the present disclosure is formed of an over-molded plastic part.

In some embodiments, a handle of a second tubular member of a catheter cutting device of the present disclosure is formed of an over-molded plastic part.

In some embodiments, a cutting head of a first tubular member of a catheter cutting device of the present disclosure is formed by laser cutting a distal end of a medical-grade hypotube.

In some embodiments, a cutting head of a first tubular member of a catheter cutting device of the present disclosure comprises a lower support arm and an upper arm having the cutting blade, wherein the upper arm is configured to move from an open position to a closed positioned relative to the lower support arm.

In some embodiments the biocompatible light curable liquid monomer of the kit is a unit dose of a light sensitive liquid monomer. In some embodiments, the introducer catheter has an inner void for passing the light-sensitive liquid to the expandable balloon, and one or more inner lumens, such as for passing a light-sensitive liquid. In some embodiments, the light-sensitive liquid is housed in a syringe. In some embodiments, the kit further includes an optical fiber, wherein the optical fiber is sized to pass through an inner lumen of the introducer catheter to guide a light into the expandable balloon to illuminate and cure the light-sensitive liquid. In some embodiments, an attachment system communicates light energy from a light source to the optical fiber.

All patents, patent applications, and published references cited herein are hereby incorporated by reference in their entirety. It will be appreciated that several of the above-disclosed and other features and functions, or alternatives thereof, may be desirably combined into many other different systems or applications.

We claim:

1. A catheter cutter device comprising:
a first tubular member having a proximal end, a distal end, and a lumen extending therethrough,
wherein the proximal end of the first tubular member includes a threaded shank portion and a plain shank portion,
wherein the plain shank portion of the first tubular member is configured to connect the first tubular member into a chuck of a drill, and
wherein the distal end of the first tubular member terminates in a cutting head comprising a cutting blade; and
a second tubular member having a proximal end, a distal end, and a lumen extending therethrough,
wherein the proximal end of the second tubular member includes a handle having internal threads.

2. The catheter cutter device of claim 1, wherein the first tubular member and the second tubular member are manufactured from a medical-grade hypotube.

3. The catheter cutter device of claim 1, wherein the proximal end of the first tubular member is formed of an over-molded plastic part.

4. The catheter cutter device of claim 1, wherein the handle of the second tubular member is formed of an over-molded plastic part.

5. The catheter cutter device of claim 1, wherein the cutting head of the first tubular member is formed by laser cutting a distal end of a medical-grade hypotube.

6. The catheter cutter device of claim 1, wherein the cutting head of the first tubular member comprises a lower support arm and an upper arm having the cutting blade, wherein the upper arm is configured to move from an open position to a closed position relative to the lower support arm.

7. The catheter cutter device of claim 1, wherein the first tubular member is coaxially disposed within the lumen of the second tubular member so that the cutting head at the distal end of the first tubular member extends beyond the distal end of the second tubular member.

8. The catheter cutter device of claim 7, wherein, when the cutting head at the distal end of the first tubular member extends beyond the distal end of the second tubular member, the threaded shank portion of the first tubular member can mate with the internal threads of the handle of the second tubular member to form a secure mechanical connection.

9. The catheter cutter device of claim 8, wherein, when the secure mechanical connection between the first tubular member and the second tubular member is formed, and the plain shank portion of the first tubular member is engaged with a drill, the first tubular member is configured to rotate and the second tubular member is configured to move along the first tubular members axis in a linear direction while not rotating.

10. A method comprising:
providing a catheter cutter device comprising:
a first tubular member having a proximal end, a distal end, and a lumen extending therethrough, wherein the proximal end of the first tubular member includes a threaded shank portion and a plain shank portion, and wherein the distal end of the first tubular member terminates in a cutting head comprising a cutting blade; and
a second tubular member having a proximal end, a distal end, and a lumen extending therethrough, wherein the proximal end of the second tubular member includes a handle having internal threads, wherein the first tubular member is coaxially disposed within the lumen of the second tubular member so that the cutting head at the distal end of the first tubular member extends beyond the distal end of the second tubular member,
wherein the threaded shank portion of the first tubular member mates with the internal threads of the handle of the second tubular member to form a secure mechanical connection;
positioning the cutting head of the catheter cutter device at a junction between an introducer catheter and a hardened expandable balloon, wherein the introducer catheter is coaxially disposed within the lumen of the first tubular member of the catheter cutter device;
connecting a drill to the plain shank portion of the first tubular member;
powering on the drill causing the first tubular member to rotate and the second tubular member to move along the first tubular members axis in a linear direction while not rotating, the linear movement of the second tubular member pressing down on the cutting head of the first tubular member so the cutting blade punctures the introducer catheter as the cutting blade rotates around the introducer catheter; and
separating the introducer catheter from the hardened expandable balloon.

11. The method of claim 10, wherein the first tubular member and the second tubular member are manufactured from a medical-grade hypotube.

12. The method of claim 10, wherein the proximal end of the first tubular member is formed of an over-molded plastic part.

13. The method of claim 10, wherein the handle of the second tubular member is formed of an over-molded plastic part.

14. The method of claim 10, wherein the cutting head of the first tubular member is formed by laser cutting a distal end of a medical-grade hypotube.

15. The method of claim 10, wherein the cutting head of the first tubular member comprises a lower support arm and an upper arm having the cutting blade, wherein the upper arm is configured to move from an open position to a closed position relative to the lower support arm.

16. The method of claim 10, wherein, when starting the drill, the handle of the second tubular member is held by a user to prevent rotation of the second tubular member when the first tubular member is rotated.

17. The method of claim 10, further comprising pulling back the catheter cutter device away from the separated hardened expandable balloon.

18. The method of claim 17, wherein, when pulling back the catheter cutter device away from the separated hardened expandable balloon, the lumen of the first tubular member includes the cut introducer catheter.

19. A kit comprising:
an expandable balloon catheter;
a biocompatible light curable liquid monomer; and
a catheter cutter device, the catheter cutter device comprising:
a first tubular member having a proximal end, a distal end, and a lumen extending therethrough, wherein the proximal end of the first tubular member includes a threaded shank portion and a plain shank portion, wherein the plain shank portion of the first tubular member is configured to connect the first tubular member into a chuck of a drill, and wherein the distal end of the first tubular member terminates in a cutting head comprising a cutting blade; and
a second tubular member having a proximal end, a distal end, and a lumen extending therethrough, wherein the proximal end of the second tubular member includes a handle having internal threads, and wherein the threaded shank portion of the first tubular member is configured to mate with the internal threads of the handle of the second tubular member to form a secure mechanical connection.

20. The kit of claim 19 further comprising instructions for use.
